(19) United States
(12) Patent Application Publication
(10) Pub. No.: US 2002/0064517 A1
(43) Pub. Date: May 30, 2002

(54) FIBRIN SEALANT AS A TRANSFECTION/TRANSFORMATION VEHICLE FOR GENE THERAPY

(76) Inventor: STEWART A. CEDERHOLM-WILLIAMS, OXFORD (GB)

Correspondence Address: T R FURMAN, BRISTOL-MYERS SQUIBB COMPANY, 100 HEADQUARTERS PARK DRIVE, SKILLMAN, NJ, 08558

(21) Appl. No.: 09/334,325

(22) Filed: Jun. 16, 1999

Related U.S. Application Data
(63) Continuation of application No. 09/303,377, filed on Apr. 30, 1999, now abandoned.
(60) Provisional application No. 60/083,571, filed on Apr. 30, 1998.
(60) Provisional application No. 60/089,543, filed on Jun. 17, 1998.

(51) Int. Cl. A61K 48/00

(52) U.S. Cl. 424/93.1; 514/44; 435/69.1; 435/325; 435/252.3; 435/458; 435/455

(57) ABSTRACT

Provided, for example, is a method of transforming a cell comprising the steps of: applying a transformation effective amount of a nucleic acid to the cell; applying a fibrin gel to the cell so as to entrap a transformation effective amount of the nucleic acid; and transforming the cell with the nucleic acid. In one aspect, the nucleic acid is applied in admixture with a fibrin or fibrinogen composition that forms the fibrin gel.

This application relates to the use of fibrin polymers as vehicles for delivering genetic material to a cell or tissue.

Fibrin sealants are used to create solid formulations of polymerized fibrin or other fibrin-related molecules. The form of the polymerization is typically initially the formation of stabile non-covalent associations between the fibrin molecules. In many cases, the non-covalent associations are supplemented by subsequent covalent cross-links that form due to the action of activated Factor XIII. These solid formulations are often used to stop or reduce fluid leakage after injury, such as air leakage from the lung or blood leakage.

One form of fibrin sealant overcomes concerns with the safety of blood products and the enzymes typically used to convert fibrinogen to a form capable of polymerization by using recombinant or autologous fibrinogen and keeping snake-derived converting enzymes well segregated from the step at which the sealant is applied to a patient. See, e.g., Edwardson et al., U.S. Pat. No. 5,739,288. Autologous fibrinogen is practical through the technology of Edwardson et al., since preparation of the sealant is conducted within minutes from a small volume of blood; and segregation of the converting enzymes is possible by stabilizing the fibrin in soluble form while affinity binding techniques are used to segregate the enzymes away from the sealant.

In gene therapy, one seeks to transfect or transform cells of a certain cell type, such as liver cells, pancreatic cells, lung cells, muscle cells, leucocytes and the like, to insert an gene to correct a genetic defect or otherwise provide a helpful function. Such a gene can include a nucleic acid construct that expresses an antisense RNA to interfere in the expression of a certain mRNA or one or more constructs that express two complementary strands designed to interfere in the expression of a certain mRNA.

Similarly, nucleic acid-based vaccines seek to induce a percentage of cells to produce immune-reaction inducing polypeptides, to induce an antibody-based or cellular-based immune response.

Where viral vectors are used in gene therapy, tissue specificity can be provided by the cell surface markers utilized by the virus to gain entry into the cells. However, this avenue is only helpful where a virus that targets a given tissue exists and can be practically utilized as a vector. Moreover, where viral vectors are known to be favorable for gene therapy, such as the adenovirus, their preferred cellular targeting mechanism may not be appropriate for the desired target cell types. Thus, further tools are needed to help increase specificity for the desired target cell type, and to overcome vector preferences for alternative cell targets.

Now provided are compositions of fibrin sealants that incorporate recombinant vectors for delivery to a tissue or cell against which the sealant will be polymerized and, typically, adhered. By use of such compositions, the vectors can be maintained at a locally at high concentration in the solid gel produced by the sealant, thereby increasing the efficiency of transfection or transformation of cells. Moreover, the fibrin gel is tolerant of a number of agents used as adjuvants in the transfection or transformation of cells. Thus, such fibrin sealant compositions can be used to deliver vectors to cells or tissues, whether or not the sought for transfection or transformation is in connection with traditional concepts of gene therapy. The method can be used, for example, to create cells, such as plant cells, that produce a desirable product (such as a protein or a small molecule produced as a result of the transforming event).

### SUMMARY OF THE INVENTION

In one embodiment, the invention provides a method of transforming a cell comprising the steps of: applying a transformation effective amount of a nucleic acid to the cell; applying a fibrin gel to the cell so as to entrap a transformation effective amount of the nucleic acid; and transforming the cell with the nucleic acid. In one aspect, the nucleic acid is applied in admixture with a fibrin or fibrinogen composition that forms the fibrin gel.

In another embodiment, the invention provides a method of conducting gene therapy comprising: conducting the steps outlined above; and implanting the transformed cells into an animal. In one aspect, the cell to which the nucleic acid is applied is a precursor of a more specialized cell type, and the method further comprises: maturing the cell to the specialized cell type either in vitro or in vivo following the implanting.

In another embodiment, the invention provides a method of conducting gene therapy comprising the steps of: applying a transformation effective amount of a gene therapy effective nucleic acid to a tissue; applying a fibrin gel to the tissue so as to entrap a transformation effective amount of the nucleic acid; and transforming cells of the tissue with the nucleic acid. In one aspect, the method further comprises: surgically exposing the tissue to allow for the applying steps.

In still another embodiment, the invention provides a method of conducting surgery on an animal comprising: surgically exposing an internal tissue; applying a transformation effective amount of a nucleic acid to a tissue; applying a fibrin gel to the tissue so as to entrap a transformation effective amount of the nucleic acid; and transforming cells of the tissue with the nucleic acid, wherein the nucleic acid encodes antigens or contains peptides that induce an antibody or cytotoxic T lymphocyte response to infection by a pathogenic microbe. In one aspect, the nucleic acid encodes antigens or contains peptides that induce an antibody or cytotoxic T lymphocyte response to infection by a pathogenic microbe that is a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia, Mycoplasma, Helicobacter, Saccharomyces, Kluveromyces, Candida, or Pneumocytis.

In yet another embodiment, the invention provides a kit comprising: (a) a first composition for forming a fibrin gel comprising one of (i) fibrin monomer, (ii) fibrinogen or another fibrin precursor or (ii) a fibrin-analog; (b) a second composition for forming a fibrin gel comprising (1), where the first composition is pursuant to (i), an agent that reverses the conditions which stabilize fibrin as the monomer, (2), where the first composition is pursuant to (ii), an agent that converts the fibrinogen or fibrin-precursor to fibrin or (3), where the first composition is pursuant to (iii), a fibrin-related molecule that forms a gel with the fibrin-analog; and (c) composed separately in a third composition or incorporated into the first or second composition, a gene therapy effective amount of nucleic acid, wherein the fibrin gel formed of the first and second compositions is effective to entrap the nucleic acid in the vicinity of a cell or tissue. In one aspect, the nucleic acid is composed with a separate adjuvant for increasing the efficacy with which the nucleic acid transforms or transfects cells.

In yet still another embodiment, the invention provides a method of conducting gene therapy comprising: transforming or transfecting cells with a nucleic acid to create recombinant cells; implanting the recombinant cells into an animal; and applying a fibrin gel to entrap recombinant cells at a desired location within the animal. In one aspect, the method further comprises: surgically exposing the tissue to allow for the implanting and applying steps.

Fibrin and Blood Clotting

One mechanism for hemostasis, i.e., prevention of blood loss, is the formation of a blood clot. Clot formation in humans occurs by means of a complex cascade of reactions with the final steps being the conversion of fibrinogen by thrombin, calcium ions and activated Factor XIII to form ultimately cross-linked fibrin II polymer, alternatively known as insoluble fibrin II polymer, which is the insoluble fibrin clot.

Fibrinogen represents about 2 to 4 grams/liter of the blood plasma protein and is a complex protein consisting of three pairs of disulfide-linked polypeptide chains designated (Aα)₂, (Bβ)₂, and γ₂. “A” and “B” represent two small amino terminal peptides, known as fibrinopeptide A and fibrinopeptide B, respectively. The six polypeptide chains of fibrinogen are folded into at least three globular domains in a linear disposition, two terminal “D-domains” and a central “E-domain”. The E-domain is believed to contain all six N-terminal residues of the polypeptide chains in fibrinogen molecule. Each D-domain contains the C-terminal sequence from one β-chain, one β-chain, and one γ-chain.

The formation of insoluble fibrin clots (e.g., cross-linked fibrin II polymer) is believed to begin with fibrinogen being converted by thrombin to fibrin I monomer.

This conversion involves thrombin-mediated cleavage of the 16 amino acid fibrinopeptide A (G1-R16) from each the two Aβ-chains of fibrinogen, producing two a-chains each with a new N-terminal having the amino acid sequence G17-P-R-V20-. The fibrin I monomer, it is believed, can spontaneously polymerize with other fibrin I or fibrin II monomers due to intermolecular interactions (i.e., non-covalent bonds) between the E-domain of the converted fibrin monomer, which now has accessible non-covalent bonding sites, and a D-domain of a different fibrin I or fibrin II monomer. Each D-domain of a fibrin monomer carries a polymerization site capable of stably interacting with an E-domain of a fibrin I or fibrin II monomer.

Contacts between the two E-domain polymerization sites of one fibrin I monomer with two complementary D-domain polymerization sites, each from two different fibrin I monomers, are believed to result in linear fibrin fibrils (i.e., polymers) with half staggered overlapping molecular contacts. The fibrin I polymer so formed is sometimes referred to as soluble fibrin I polymer because, by treatment with appropriate chemical means, the fibrin I polymer can be depolymerized and reconverted to fibrin I monomers.

The next step in the formation of fibrin clots involves the conversion of fibrin I monomer to fibrin II monomer. This step involves the thrombin-mediated cleavage of the fibrinopeptide B from each of the two Bβ-chains of fibrin I. The removal of the 14 amino acid fibrinopeptide B produces β-chains, each having a N-terminal sequence of G-H-R-. Fibrin II monomers, like fibrin I monomers, can spontaneously polymerize with other fibrin II or fibrin I monomers due to intermolecular interaction sites in the E-domain of one fibrin II monomer, which are made accessible by the cleavage reaction, with the D-domain of another fibrin II or fibrin I monomer. Like fibrin I polymer, fibrin II polymer is also sometimes referred to as soluble fibrin II polymer because by use of appropriate chemical treatments it can be depolymerized and reconverted to fibrin II monomers. The exposure of the β-chain N-terminal sequences in the E-domain is important to fibrin clot formation as it facilitates covalent crosslinking by activated Factor XIII of adjacent fibrin II monomers in the fibrin II polymer. Although activated Factor XIII is also capable of crosslinking fibrin I monomers in a fibrin polymer, the reaction is less efficient due to the presence of fibrinopeptide B on fibrin I. Cross-linked fibrin II polymer is sometimes referred to as insoluble fibrin II polymer because it cannot be depolymerized and reconverted to fibrin II monomers .

In addition to thrombin and Factor XIII, calcium ions are believed to be important in the formation of fibrin clots and have a number of important roles. Calcium ions are believed necessary for the activation of prothrombin to thrombin, and since thrombin activates Factor XIII, calcium ions are indirectly necessary for Factor XIII activation. Further, active Factor XIII is believed to be a calcium-dependent enzyme that cannot cross-link fibrin polymers in the absence of calcium ions. Calcium ions also directly bind to polymeric fibrin and change the opacity and mechanical properties of the fibrin polymeric strands. For reviews of the mechanism of blood coagulation and the components of a fibrin clot, see C. M. Jackson, Ann. Rev. Biochem., 49:765-811, 1980, and B. Furie and B. C. Furie, Cell, 53:505-518, 1988.

Fibrin Sealants

A fibrin sealant is a biological adhesive whose effects imitate the stages of coagulation to form a fibrin polymer. The sealant can be designed so that the fibrin monomer will be converted to insoluble fibrin polymer. One type of fibrin sealant uses fibrinogen and consists of two components. One component comprises concentrated human fibrinogen, bovine aprotinin and Factor XIII. The second component comprises calcium chloride and an enzyme, such as thrombin, that converts fibrinogen to fibrin. Application of this type of sealant is generally carried out with a double-barreled syringe, which permits simultaneous delivery of both components to the desired site of the fibrin clot formation. The mixing of the two components at the target site produces a fibrin clot via the sequence of reactions described above.

The fibrinogen component of this type of fibrin sealant is typically prepared from pooled human plasma. The fibrinogen can be concentrated from the human plasma by cryoprecipitation and precipitation using various reagents, e.g., poly(ethylene glycol), diethyl ether, ethanol, ammonium sulfate or glycine. For reviews of this type of fibrin sealants, see M. Brennan, Blood Reviews 5:240-244, 1991; J. W. Gibble and P. M. Ness, Transfusion 30:741-747, 1990; H. Matras, J. Oral Maxillofac. Surg. 43:605-611, 1985 and R. Lemer and N. Binur, J. of Surgical Research 48:165-181, 1990.

A second, newer type of fibrin sealant uses compositions consisting primarily of fibrin I or fibrin II monomers. See European Patent Application No. 0 592 242, published April, 1994. In these types of sealants, fibrin I monomers or fibrin II monomers or desBB fibrin monomers are prepared in advance of sealant application from fibrinogen using an appropriate proteolytic enzyme, such as thrombin or batroxobin. The fibrin monomers are maintained in soluble form using an appropriate buffer. Useful buffers include those that have a low pH or a chaotropic agent. The fibrin I monomers, fibrin II monomers or desBB fibrin monomers in such solutions can be converted to fibrin polymers by mixing the solution with a second solution to produce a mixture with conditions that permit the spontaneous polymerization of the fibrin monomers to form a fibrin clot.

Fibrin I, fibrin II and desBB fibrin monomer-based sealants have several advantages over fibrinogen-based sealants. Notably, fibrin monomer-based sealants do not include bovine or human thrombin. The use of such sealants, when the fibrin monomer is prepared from the autologous source (i.e., the patients themselves), introduces no foreign proteins into the recipient and thereby avoids complications arising from immunological reactions and risk of blood-borne infections. The fibrin monomer-based sealants can be conveniently prepared. Soluble fibrin polymer can be dissolved using a weak acidic solution. In some embodiments, the resulting fibrin monomers are lyophilized to fine powders. Such powders can easily be re-dissolved in a weak acid and induced to re-polymerize by the addition of an alkali buffer. Alternatively, the powdered fibrin monomers can be dissolved in a chaotropic solution, e.g., urea, to a high concentration (>150 mg/ml) and induced to re-polymerize by the addition of water.

A further advantage of fibrin monomer-based sealants is that as they generally use autologous components, their use poses a lower risk of exposure to blood-transmitted infectious agents such as hepatitis (including hepatitis B, and non-A, non-B hepatitis) and acquired immune deficiency virus (AIDS). See L. E. Silberstein et al., Transfusion, 28:319-321, 1988; K. Laitakari and J. Luotonen, Laryngoscope 99:974-976, 1989; and A. Dresdale et al., Annals of Thoracic Surgery 40:385-387, 1985. Diseases caused by such agents can be transmitted by conventional fibrinogen-based sealants because the fibrinogen component is typically prepared from pooled human plasma. Moreover, the use of fibrin-based sealants can also avoid the risks associated with the bovine thrombin component of fibrinogen-based sealants. Bovine thrombin preparations can carry the infectious agent bovine spongiform encephalitis (BSE) as well as viral pathogens of mammals. Also, bovine thrombin is a potent antigen, which can cause adverse immunological reactions in humans. For further discussions of these types of complications that are associated with fibrinogen-based sealants, see Taylor, J. Hospital Infection 18 (Supplement A):141-146, 1991 and Prusiner et al., Cornell Vet 81:85-96, 1991.

Recombinant Fibrinogen and Fibrin

Genetic engineering can produce fibrinogen and fibrin monomers in comparatively high yields, in substantially pure form, and in the absence of pathogenic viruses such as hepatitis and HIV. Heterologous expression of fibrinogen and fibrin chains also allows the construction of mutations which can mimic naturally occurring fibrin variants, and the isolation and study of these proteins without a need for patients with these rare genetic defects.

Each of the three different polypeptide chains (Aα, Bβ and γ) of fibrinogen is coded by a separate gene. The cDNAs for each of these chains have been prepared (Chung et al., Ann. N. Y Acad. Sci. 408:449-456, 1983; Rixen et al., Biochemistry 22:3237-3244, 1983; Chung et al., Biochemistry 22:3244-3250, 1983; Chung et al., Biochemistry 22:3250-3256, 1983) and expressed in prokaryotic organisms. Furthermore, each human fibrinogen chain has been introduced separately (Huang et al., J. Biol. Chem. 268:8919-8926, 1993; Roy et al., J. Biol. Chem. 267:23151-23158, 1992; Roy et al., J. Biol. Chem. 266:4758-4763, 1991) or in combination (Hartwig and Danishefsky, J. Biol. Chem. 266:6578-6585, 1991; Huang et al., J. Biol. Chem. 268:8919-8926, 1993; Roy et al., 1991, J. Biol. Chem., 266:4758-4763; Redman and Samar, U.S patent application Ser. No. 07/663,380, filed March, 1991, available from Natl. Technology Information Service No. PAT-APPL07663 380INZ) into expression plasmids and transfected into eukaryotic cells.

Most of the plasmids used in expressing recombinant human fibrinogen are derived from those constructed by Dr. D. Chung, University of Washington, Seattle and are based on cDNA clones (Rixen et al., Biochemistry 22:3237-3244, 1983; Chung et al., Biochemistry 22:3244-3250, 1983; Chung et al., Biochemistry 22:3250-3256, 1983). The expression of recombinant fibrinogen chains was first achieved in E. coli (Bolyard and Lord, Gene 66:183, 1988; Bolyard and Lord, Blood, 73:1202-1206, 1989; Lord and Fowlkes, Blood, 73:166-171, 1989). The individually expressed chains show antigenic similarities with fibrinogen and display thrombin cleavable sites similar to those found in native fibrinogen (Bolyard and Lord, Blood, 73:1202-1206, 1989; Lord and Fowlkes, Blood, 73:166-171, 1989). Fibrinopeptides A and B can be released from recombinant fibrinogen (Bolyard and Lord, Blood, 73:1202-1206, 1989; Lord and Fowlkes, Blood, 73:166-171, 1989).

Eukaryotic cells carrying appropriate expression plasmids encoding individual fibrinogen chains have been shown to synthesize the encoded fibrinogen chains and to result in the intracellular formation of dimeric chain molecules, e.g. Aα₂, Bβ₂ or γ₂ dimers (Roy et al., J. Biol. Chem., 265:6389-6393, 1990; Zhang and Redman, J. Biol. Chem. 267:21727-21732, 1992). Furthermore, when appropriate plasmids containing genes encoding for all three human fibrinogen chains are transferred into the same cell, then not only are all three chains expressed but the polypeptide chains associate in pairs and intact fibrinogen is secreted into the surrounding medium (Roy et al., J. Biol. Chem., 266:4758-4763, 1991; Hartwig and Danishefsky, J. Biol. Chem. 266:6578-6585, 1991). Like natural fibrinogen, the secreted recombinant fibrinogen consists of three pairs of non-identical polypeptide chains and is functional in forming fibrin polymers.

Fibrinogen is naturally synthesized by liver, and megakaryocyte cells and transformed liver cells maintained in culture are able to continue fibrinogen synthesis and secretion (See Otto et al., J. Cell. Biol. 105:1067-1072, 1987; Yu et al., Thromb. Res. 46:281-293, 1987; Alving et al., Arch. Biochem. Biophys. 217:19, 1982). One such cell line is the Hep G2 cells (Drs. Knowles and Aden, Wister Institute, Philadelphia). This line synthesizes an excess of Aα- and γ-chains over the Bb-chains resulting in nonproductive dimeric complexes of Aα- and γ-chains (e.g., Aα2γ2). The introduction of an additional expression vector encoding Bβ-chains resulted in the formation of trimeric complexes (AαBβγ) which adopt the correct folding and intrachain disulfide bonding patterns (Roy et al., J. Biol. Chem., 265:6389-6393, 1990). The mechanism of this folding is unknown and may involve ancillary proteins and enzymes (Roy et al., J. Biol. Chem., 267:23151-23158, 1992). These studies demonstrated not only the correct transcription of Bβ cDNA but also that the excess Bβ-chain enhanced the assembly and secretion of intact fibrinogen.

In Hep G2 cells, the AαBβγ trimeric complexes associate in pairs to form intact fibrinogen molecules, which become glycosylated and are actively secreted from the cell (Huang et al., J. Biol. Chem. 268:8919-8926, 1993). Indeed only correctly assembled fibrinogen molecules are secreted. Thus, Hep G2 cells have the synthetic and secretory apparatus for the assembly of fibrinogen.

Subsequent experiments have introduced fibrinogen chain encoding cDNA plasmids into eukaryotic cells that do not normally synthesize fibrinogen. These experiments successfully produced functional fibrinogen, demonstrating that the factors needed for fibrinogen assembly and secretion are not unique to liver-derived cells like Hep G2. Eukaryotic cells known to be capable of assembling and secreting recombinant fibrinogen include baby hamster kidney cells (BHK), COS cells and Chinese hamster ovary cells (CHO) (Roy et al., J. Biol. Chem. 266:4758-4763, 1991; Hartwig and Danishefsky, J. Biol. Chem. 266:6578-6585, 1991; Farrell et al., Biochemistry 30:94149420, 1991).

Intact functional fibrinogen secreted by stably transformed eukaryotic cells results in the accumulation of fibrinogen levels of around 1-2 μg/ml. Methods are known for increasing the output of recombinant proteins from transfected cells like CHO cells such that the expression levels can approach a thousand fold the basal secretory level.

Additional description of methods of recombinantly producing fibrin-related molecules can be found in PCT/US95/05527.

Gene Therapy

A lesson of the last 20 plus years in which scientists have begun actively considering methods to introduce genetic material into appropriate target tissues to overcome a genetic disease has been that the effort is more complex than was initially anticipated. Some of the goals needed to be met to create successful gene therapy tools include: (1) efficient transduction of the target cells; (2) long-term expression of the gene; (3) lack of a disabling immune response to the vector or transduced cell; and (4) absence of toxicity. See, Samulski et al., “Adeno-associated Viral Vectors” in Development of Human Gene Therapy, Cold Spring Harbor Laboratory Press, 1998, pp. 131-172. (This article, along with this entire treatise on gene therapy, is incorporated by reference herein.) All the above listed goals, especially the first three, identify areas that have given rise to substantial barriers to efficient gene therapy. Vectors typically transduce only a percentage of the cells to which they are applied. The transducing gene is often maintained on an episome and is therefore often not a stably incorporated and maintained genetic element. Moreover, incorporation into the chromosomal DNA is often dependent on cell division, thereby limiting the scope of target tissues to replicating tissues. Viral vectors often carry the nucleic acid encode proteins that induce immunity, thereby carrying the seeds for the destruction of the transduced cells. Certain viral vectors overcome some of these problems but otherwise create at least an implication of danger. For example, non-replicating forms of the human immunodeficiency virus are being engineered for use as gene therapy vectors that allow for the incorporation of the genetic material into genomic DNA. Such vectors must maintain the genetic tools by which to facilitate genomic incorporation, but must lack enough of the gene products that create infectivity, such that in this case for AIDS there is no chance that recombination events will regenerate an infective particle. See, Naldini et al., “Lentiviral Vectors” in Development of Human Gene Therapy, Cold Spring Harbor Laboratory Press, 1998, pp. 47-60.

The good news is that all of these problems are now well-recognized, and the viral vectors used in gene therapy have improved to address such problems. Moreover, gene therapy can be conducted without viral vectors. Also, in other genetic transformations the problems of toxicity and immune response do not come to fore to the same degree. In nucleic acid-based vaccines, for example, an immune response is desirable, as can be a process by which expression of the transforming gene attenuates so that production of the immuno-stimulant attenuates over time.

Viral vectors have also been subject to engineering to change their target cell preference, for instance by binding or incorporating antibodies. For instance, Valsesia-Wittmann et al. modified the cell-surface binding characteristics of avian leukosis virus. J. Virol. 68: 4609-4619, 1994. Erythropoietin, which of course binds its cognate receptor, has been incorporated into Moloney murine leukemia virus (Mo-MLV). Kasahara et al., Science 266: 1373-1376, 1994. A tumor-targeting single-chain antibody has been incorporated into spleen necrosis virus. Chu and Dornburg, J. Virol. 69: 2659-2663, 1995. HIV envelop protein has been incorporated into murine leukemia viral vectors. Mammamo et al., J. Virol. 71: 3341-3345, 1997. Such targeting methods with respect to adenoviral vectors are reviewed by Reynolds and Curiel, “Strategies to Adapt Adenoviral Vectors for Gene Therapy Applications: Targeting and Integration,” in Development of Human Gene Therapy, Cold Spring Harbor Laboratory Press, 1998, pp. 111-130.

As reviewed in Development of Human Gene Therapy, Cold Spring Harbor Laboratory Press, 1998, a wide variety of viral vectors have been selected or engineered for gene therapy. Moreover, nucleic acid can be delivered successfully without the use of viral vectors. For example, an early-developed method for increasing transfection efficiency was to use calcium phosphate-precipitated nucleic acid. The transfection potential of nucleic acid is increased by compacting it with polycationic polymers such as DEAE dextran (Veheri et al., Virology 27: 434-436, 1965), polylysine (Wu et al., J. Biol. Chem. 266: 14338-14342 1991), cationic peptides (Wadhwa et al., Bioconjugate Chem. 8: 81-88, 1997; and Niidome et al., J. Biol. Chem. 272: 15307-15312 1997), polyethyleneimine (Boussiffet al., Proc. Natl. Acad. Sci USA 92: 7297-7301, 1995), a glucaramide-based polyamino polymer (Goldman et al., Nat. Biotechnol. 15: 462-466, 1997), polyamidoamine dendrimers (Dielinska et al., Biochim. Biophys. Acta 1353: 180-190, 1997). Other polymers useful as enhancers of nucleic acid uptake include erodable microspheres (Mathiowitz et al., Nature 386: 410-412, 1997) and polyvinyl pyrrolidone (Mumper et al., Pharm. Res. 13: 701-709, 1996). Other enhancers include cationic liposomes into which the nucleic acid is incorporated. Feigner et al., 1987; Felgner and Ringold, 1989. Such liposomes, or “lipoplexes,” are believed to insert the nucleic acid into a target cell by a membrane fusion mechanism. Illustrative of the many cationic lipid formulations now available (see, Felgner et al., “Synthetic Delivery Systems,” in Development of Human Gene Therapy, Cold Spring Harbor Laboratory Press, 1998, pp. 241-260), is DOTMA (N[1-(2,3-dioleyloxy)propyl]-N,N,N-trimethylammonium). Other such cationic lipid formulations include Lipofectin™, a 1:1 (w/w) liposome formulation of the cationic lipid N-[1-(2,3-dioleyloxy)propyl]-N,N,N-trimethylammonium chloride (DOTMA) and dioleoyl phosphatidylethanolamine (DOPE), LipofectAMINE™, a 3:1 (w/w) liposome formulation of the polycationic lipid 2,3-dioleyloxy-N-[2(spermine-carboxamido)ethyl]-N,N-dimethyl-1-propanaminiumtrifluoroacetate (DOSPA) and the neutral lipid dioleoyl phosphatidylethanolamine (DOPE) in membrane-filtered water, and LipofectACE™, a 1:2.5 (w/w) liposome formulation of the cationic lipid dimethyl dioctadecylammonium bromide (DDAB) and dioleoyl phosphatidylethanolamine (DOPE) in membrane-filtered water (all from Life Technologies, Rockville, Md.). Moreover, gene transfer can also be achieved without such adjuvants. Targeting techniques can also be employed which bind or affix targeting molecules to the nucleic acid or nucleic acid complex to be used for transfection. Cotton and Wagner, “Receptor-mediated Gene Delivery Strategies,” in Development of Human Gene Therapy, Cold Spring Harbor Laboratory Press, 1998, pp. 261-277.

As will be recognized by those of ordinary skill, the nucleic acid sought to be introduced into cells will often include, in addition to the portion conveying the primary genetic characteristic of interest, a portion encoding a substance that is itself, or gives rise to, a molecule that is readily detectable. This “reporter” molecule serves as a surrogate for determining or estimating success in introducing the primary genetic characteristic. Where cells in culture are being transformed, a portion of the nucleic acid can encode a substance required for the cells to survive in the face of an appropriate challenge.

The nucleic acid can be single or double-stranded, though non-virally mediated techniques that seek to express a portion of the nucleic acid will typically use double-stranded nucleic acid.

Preparation of a Preferred Sealant Composition

First, blood is collected and a plasma is isolated. Added to the plasma is an enzyme that converts fibrinogen to fibrin. In converting fibrinogen to fibrin, preferably care is taken to prevent the formation of cross-links between fibrin molecules via the transaminase activity of factor XIII^{a}. This can be done by a number of techniques including for example the use of factor XIII^{a} inhibitors such as heavy metals (such as mercury), thiomerosal {[(o-carboxyphenyl) thio]ethyl mercury sodium salt}, inhibitory antibodies, or calcium chelators (since calcium is a necessary cofactor for the enzyme). Calcium chelators include, but are not limited to, EGTA (ethylenglycolbis-(2-aminoethylether)tetra-acetic acid), and the like. For example, the converting enzyme is batroxobin, (“Btx”), a proteinase from the snake venom of snakes of the genus Bothrops, used at a concentration of about 0.1 μg/ml to about 100 μg/ml, preferably to a concentration of about 0.5 μg/ml to about 50 μg/ml.

Other proteinases of appropriate specificity can also be used. Snake venom proteinases are particularly suitable, including without limitation the venom enzymes from Agkistrodon acutus, Agkistrodon contortrix contortrix, Agkistrodon halys pallas, Agkistrodon (Calloselasma) rhodostoma, Bothrops asper, Bothrops atrox, Bothrops insularis, Bothrops jararaca, Bothrops Moojeni, Lachesis muta muta, Crotalus adamanteus, Crotalus durissus terrificus, Trimeresurus fiavorviridis, Trimeresurus gramineus and Bitis gabonica.

The fibrinogen-converting enzyme is favorably coupled to a converting enzyme binding partner which is used in an affinity procedure to reduce the concentration of the enzyme in a preparation. In the example, the converting enzyme binding partner is biotin, a member of the biotin-avidin binding pair, a pair of molecules that bind with extremely high affinity. An amino acid sequence for avidin is described in Dayhoff, Atlas of Protein Sequence, Vol. 5, National Biomedical Research Foundation, Washington, D.C., 1972 (see also, DeLange and Huang, J. Biol. Chem. 246: 698-709, 1971), and an amino acid sequence for Streptavidin is described in Argarana et al., Nucl. Acid Res. 14:1871-1882, 1986. Nucleic acid sequences are available, for example, as follows: (1) chicken mRNA for avidin, Gene Bank Acc. No. X05343, Gore et al., Nucl. Acid Res. 15: 3595-3606, 1987; (2) chicken, strain White Leghorn gene for avidin, Gene Bank Acc. No. L27818 (3) streptavidin from Strep. avidinii, Gene Bank Acc. No. X03591, Argarana et al., Nucl. Acid Res. 14:1871-1882, 1986; (4) synthetic gene for streptavidin from Strep. avidinii, Gene Bank Acc. No. A00743, Edwards, W089/03422; and (5) synthetic gene for streptavidin, Gene Bank Acc. No. X65082, Thompson et al., Gene 136: 243-246,1993.

Avidin and Streptavidin are preferably used in a tetrameric form, although monomers can be used. Other binding pairs that bind with high affinity include an antibody specific for a polypeptide or other molecule, any polypeptide to which an antibody is available or can be prepared, thioredoxin, which binds phenylarsine oxide (expression vectors include, for example, the thioredoxin fusion protein vector pTrxFus available from Invitrogen, Carlsbad, Calif.), poly-His sequences that bind to divalent cations such as nickel II (expression vectors include, for example, the pThioHis vectors A, B and C available from Invitrogen), glutathione-S-transferase vectors that bind to glutathione (vector for example available from Pharmacia Biotech, Piscataway, N.J.). Methods of producing such antibodies are available to those of ordinary skill in light of the description herein of polypeptide expression systems and of known antibody production methods. For antibody preparation methods, see, for example, Ausubel et al., Short Protocols in Molecular Biology, John Wiley & Sons, New York, 1992. Very high affinity binding characteristics, while highly convenient, are not essential. Any affinity that can be used in an affinity-binding procedure to reduce the concentration of converting enzyme in a preparation can be used in this context. Note that if the affinity procedure simply uses an antibody against the converting enzyme, then this aspect of the invention does not require a coupled converting enzyme binding partner, since the enzyme itself comprises the converting enzyme binding partner.

Unless the process is designed to prevent polymerization of fibrin monomer during the enzymatic conversion from fibrinogen to fibrin, the fibrin formed will polymerize into fibrin polymer, and thereby form a fibrin clot. After the solids are isolated, fibrin monomer is recovered from the fibrin clot. Fibrin monomer is recovered, for example, by adding a solubilizing agent to the fibrin clot. Such solubilizing agents can include, for example, acid solutions such as aqueous solutions having pH of about 5 or less, or chaotropic agents, such as urea, sodium bromide, guanidine hydrochloride, potassium cyanide, potassium iodide or potassium bromide. The solubilizing agents can be used at near the minimum concentration effective to maintain fibrin monomer (i.e., a fibrin-solubilizing effective amount). A number of conditions for forming fibrin monomer are described in Edwardson et al., European Patent Application No. EP 592,242.

A solid material having bound thereto a second binding partner, which is the complementary binding partner to the converting enzyme binding partner, is then added the fibrin monomer preparation to bind any converting enzyme as may continue to be found in the preparation. The solids, which, depending on the protocol used, can include the solid material or any residual fibrin clot material, is then removed, for instance by filtration or centrifugation.

The processed material can be stored in liquid form, for instance at about 4° C. or less, in frozen form, or as a dried form such as a lyophilizate. Lyophilizates are formed by standard methods. These lyophilizates are generally reconstituted in purified water or in a buffered aqueous solution. For the fibrin monomer, generally, the same solution composition of solubilizing agent previously used in the process can be used to reconstitute the lyophilizate. Or, if the user desires the fibrin to polymerize on reconstitution, an aqueous solution, which either (a) lacks a solubilizing agent or (b) is capable of reversing any solubilizing conditions carried in the lyophilizate, is employed.

As illustrated, to form fibrin sealants (i.e., clots) the fibrin monomer and a non-enzymatic polymerizing agent can be mixed together. The polymerizing agent is any reagent effective to reverse the conditions that prevent the polymerization of fibrin monomer. For example, if fibrin monomer is in an acidic solution, such as a 0.2 M sodium acetate, pH 4.0 solution, the polymerizing agent can be a basic solution, such as, without limitation, a solution of HEPES (N-[2-hydroxyethyl)piperazine-N′[ethanesulfonic acid]), sodium hydroxide, potassium hydroxide, calcium hydroxide, bicarbonate buffers such as sodium bicarbonate and potassium bicarbonate, tri-metal salts of citric acid, salts of acetic acid and salts of sulfuric acid. Preferred alkaline buffers include: carbonate/bicarbonate; glycine; bis hydroxeythylaminoethane sulphonic acid (BES); hydroxyethylpiperazine propane sulphonic acid (EPPS); Tricine; morpholino propane sulphonic acid (MOPS); trishydroxymethyl aminoethane sulphonic acid (TES); cyclohexylaminoethane sulphonic acid (CHES); trishydroxymethyl aminoethane sulphonic acid (TES). The amount of alkaline buffer that is utilized should be enough to allow polymerization of the fibrin. It is preferred that about 10 parts to about one part of composition comprising fibrin monomer be mixed with about 1 part alkaline buffer. It is even more preferred that such ratio be about 9:1. The preferred ratio depends on the buffer, its concentration and pH, and the desired concentration of the fibrin polymer. Where acidic pH is used as the solubilizing agent, the fibrin solubilization can occur in the presence of calcium ions, such as at a concentration of about 20 mM.

Incorporating Nucleic Acid into the Fibrin Gel

In one preferred embodiment, three streams of aqueous preparations are mixed to initiate a rapid clot formation process. These preparations can be, for example, a fibrin monomer preparation, a composition comprising the nucleic acid for transforming or transfecting cells (“transforming composition” or “TC”), and a non-enzymatic polymerizing agent. Or, in another example, the preparations are fibrinogen, a fibrinogen-converting enzyme and the TC. To allow the resulting gel-forming mixture to remain pliable for period of time, the sealant mixture is generally formed either during the process by which the sealant is applied to its recipient surface, or within a few minutes prior to application. Generally, the sealant mixture remains conveniently pliable for about 30 seconds or less.

In another preferred embodiment, the three streams are sprayed so that they converge and mix. Suitable spray heads are described in U.S. Pat. Nos. 5,605,541, 5,376,079, and 5,520,658 and PCT Application 97/20585. Where the spray heads utilized are designed to spray only one solution, additional spray heads can be aligned to deliver other solutions to the site of delivery. For example, where the spray head delivers two concentric rings of sprayed solution or suspension, and uses gas outlets to shape and merge the streams, a second such spray head can be used to deliver a third solution.

Instead of three streams, the TC can, where appropriate, be incorporated into one of the other two preparations.

Alternatively, the TC can be mixed with the sealant after the polymerization process has been initiated but while the composition remains pliable. Or, the TC can be applied to cells or tissue, and the sealant can be applied to fix the TC in place. Such a subsequently applied sealant would preferably be applied concurrent with or soon after the process which polymerizes the sealant is initiated.

Other types of fibrin sealant useful in the invention, other than that described in some detail above, are described in, for example: Wadtröm, U.S. Pat. No. 5,631,011; Cochrum, U.S. Pat. No. 5,510,102; Pines et al., U.S. Pat. No. 5,330,974; Matras, J. Oral Maxillofacial Surgery 13: 605-611, 1985; and Brennan, Blood Reviews 5: 240-244, 1991. Another device for spraying a fibrin sealant is described, for example, in Avoy, U.S. Pat. No. 4,902,281.

Miscellaneous Aspects

When body fluids are used as the source for fibrin, in many cases it will be desirable to isolate with the fibrin ancillary factors such as factor XIII or factor XIII^{a} and thrombin. When purification techniques are used that isolate fibrin via the reversible formation of a fibrin polymer, it is believed that the fibrin polymer has affinity for a number of such ancillary factors, such that the isolated product will retain these factors. In some cases, it will be desirable to limit the amount that non-fibrin materials are washed out of the fibrin polymer, for instance, by limiting the degree to which the fibrin polymer is compressed in the course of a method according to the invention, in order to assure the co-isolation of sufficient amounts of ancillary factors.

The present invention can be used for treating any animal having a fibrin-based system for controlling bleeding, but is preferably used for treating mammals, most preferably humans.

Definitions

The following terms shall have, for the purposes of this application, the respective meaning set forth below.

cell precursor of a more specialized cell type. A precursor cell is a cell, typically referred to as a “stem” cell or a “pluripotent” cell, which has the potential to, but has not yet, differentiated into a more specialized cell.

fibrin. One of a number of derivatives of fibrinogen {e.g., fibrin I (i.e., desAA-fibrin), fibrin II (i.e., desAAdesBB fibrin) or des BB fibrin} that can polymerize to form a precipitate of fibrin polymer. The derivatives are typically created by cleaving the A or B fibrinopeptides from fibrinogen.

fibrin analog. A form of fibrin monomer is an engineered version of fibrin, or “fibrin analog,” which will not self-polymerize, but will polymerize with another fibrin-related molecule such as fibrinogen. Such an engineered fibrin is described in Cederholm-Williams et al., “Recombinant Fibrin Chains, Fibrin and Fibrin-Homologs,” PCT Application No. PCT/US95/05527, filed May 2, 1995.

fibrin chain precursor. Precursor of a fibrin α-chain or α-chain containing a N-terminal peptide that can be cleaved to yield the fibrin chain effective in a fibrin to allow polymerization.

fibrin clot-forming effective amount. An effective amount of clot-forming fibrin is that quantity or concentration (if in a liquid form) of a fibrin (for example fibrin monomer) which forms sufficient clot material to be of utilized as a fibrin sealant.

fibrin monomer. Fibrin monomer is fibrin that is held in soluble form and prevented from clotting, for instance by the presence of a polymerization inhibitor such as acidic pH or a chaotropic agent or by being kept in a form which prevents polymerization, such as a sufficiently dehydrated form or a frozen form.

fibrin polymer. Fibrin molecules, in the absence of conditions that prevent polymerization of fibrin monomer, interact noncovalently to form polymers, here termed “fibrin polymers”, which—when sufficient mass is achieved —form a visible adherent precipitate with clot-like properties. By the action of factor X_{III}^{a}, fibrin polymer can be covalently crosslinked. Prior to the crosslinking action of factor XIII^{a}, fibrin polymer can be reversibly converted to fibrin monomer. Even when some initial such crosslinking has occurred, it is believed that fibrin polymer can be reversibly converted to fibrin monomer.

fibrin precursor. A Precursor of fibrin comprises one or more fibrin chain precursors which must be processed to yield a form of fibrin that polymerizes with corresponding fibrin molecules. A fibrin precursor contains one or more leader peptides on its constituent chains. The leader peptide(s) can be processed in vivo or in vitro from the fibrin precursor to yield fibrin.

gene therapy. As used herein, “gene therapy” includes any intervention in an animal (preferably a mammal, more preferably a human) that (i) causes a cell in the animal to express (as RNA or protein) a recombinant nucleic acid, whether such expression is transient or stable, or (ii) causes a change in the cell's genome, such as an insertion, that changes the cell's pattern of gene expression. Hence, gene therapy includes uses of nucleic acid-based vaccines.

high affinity binding. High affinity binding between a first substance and a second substance is binding of sufficient avidity to allow for the first or second substance to be used as an affinity ligand for the isolation of the other substance. Typically, high affinity binding is reflected in an association constant of about 10⁵ M⁻¹ or more, preferably 10⁶ M⁻¹ or more, yet more preferably 10⁷ M⁻¹ or more.

Or. The conjunction “or” is used to express that at least one of the recited alternatives linked by or is applicable in a given context and to include the conjunctive sense , joining two or more of the recited alternatives. In other words, unless the context indicates a contrary meaning, “or” includes the meaning sometimes expressed as “and/or.”

Polynucleotide or nucleic acid. The terms polynucleotide(s) or nucleic acid(s) (herein “polynucleotide(s)”) generally refer to any polyribonucleotide or polydeoxyribonucleotide, which can be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that can be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

transformed cell. A cell is transformed if a nucleic acid is recombinantly introduced into it or its ancestor so as to temporarily or stably (1) cause the cell to express a polypeptide or RNA in an amount not otherwise expressed by the cell or (2) interfere with the translation or transcription of a nucleic acid normally found in the cell.

transforming composition. A transforming composition is a composition containing a gene therapy effective amount of a nucleic acid.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of transforming a cell comprising the steps of:
applying a transformation effective amount of a nucleic acid to the cell;
applying a fibrin gel to the cell so as to entrap a transformation effective amount of the nucleic acid; and
transforming the cell with the nucleic acid.

2. The method of claim 1, wherein the nucleic acid is applied in admixture with a fibrin or fibrinogen composition that forms the fibrin gel.

3. A method of conducting gene therapy comprising:
conducting the steps of claim 1; and
implanting the transformed cells into an animal.

4. The method of claim 3, wherein the cell to which the nucleic acid is applied is a precursor of a more specialized cell type, and the method further comprises:
maturing the cell to the specialized cell type either in vitro or in vivo following the implanting.

5. A method of conducting gene therapy comprising the steps of:
applying a transformation effective amount of a gene therapy effective nucleic acid to a tissue;
applying a fibrin gel to the tissue so as to entrap a transformation effective amount of the nucleic acid; and
transforming cells of the tissue with the nucleic acid.

6. The method of claim 5, further comprising:
surgically exposing the tissue to allow for the applying steps.

7. A method of conducting surgery on an animal comprising:
surgically exposing an internal tissue;
applying a transformation effective amount of a nucleic acid to a tissue;
applying a fibrin gel to the tissue so as to entrap a transformation effective amount of the nucleic acid; and
transforming cells of the tissue with the nucleic acid, wherein the nucleic acid encodes antigens or contains peptides that induce an antibody or cytotoxic T lymphocyte response to infection by a pathogenic microbe.

8. The method of claim 7, wherein the nucleic acid encodes antigens or contains peptides that induce an antibody or cytotoxic T lymphocyte response to infection by a pathogenic microbe that is a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia, Mycoplasma, Helicobacter, Saccharomyces, Kluveromyces, Candida, or Pneumocytis.

9. A kit comprising:
(a) a first composition for forming a fibrin gel comprising one of (i) fibrin monomer, (ii) fibrinogen or another fibrin precursor or (ii) a fibrin-analog;
(b) a second composition for forming a fibrin gel comprising (1), where the first composition is pursuant to (i), an agent that reverses the conditions which stabilize fibrin as the monomer, (2), where the first composition is pursuant to (ii), an agent that converts the fibrinogen or fibrin-precursor to fibrin or (3), where the first composition is pursuant to (iii), a fibrin-related molecule that forms a gel with the fibrin-analog; and
(c) composed separately in a third composition or incorporated into the first or second composition, a gene therapy effective amount of nucleic acid, wherein the fibrin gel formed of the first and second compositions is effective to entrap the nucleic acid in the vicinity of a cell or tissue.

10. The kit of claim 9, wherein the nucleic acid is composed with a separate adjuvant for increasing the efficacy with which the nucleic acid transforms or transfects cells.

11. A method of conducting gene therapy comprising:
transforming or transfecting cells with a nucleic acid to create recombinant cells;
implanting the recombinant cells into an animal; and
applying a fibrin gel to entrap recombinant cells at a desired location within the animal.

12. The method of claim 11, further comprising:
surgically exposing the tissue to allow for the implanting and applying steps.
